(19) United States
(12) Patent Application Publication
(10) Pub. No.: US 2025/0312246 A1
(43) Pub. Date: Oct. 9, 2025

(54) ZIRCONIA MODIFICATION SOLUTION AND USE THEREOF

(71) Applicant: AIDITE (QINHUANGDAO) TECHNOLOGY CO., LTD., Qinhuangdao (CN)

(72) Inventors: Haiyan WU, Qinhuangdao (CN); Shenggang ZHOU, Qinhuangdao (CN); Xiaoyu LI, Qinhuangdao (CN); Yang GAO, Qinhuangdao (CN)

(21) Appl. No.: 19/242,005

(22) Filed: Jun. 18, 2025

Related U.S. Application Data
(63) Continuation-in-part of application No. PCT/CN2024/127719, filed on Oct. 28, 2024.

(30) Foreign Application Priority Data
Oct. 30, 2023 (CN) 202311420248.9

(51) Int. Cl. A61K 6/818; A61C 13/083; A61K 6/60; A61K 6/78

(52) U.S. Cl. CPC A61K 6/818; A61C 13/083; A61K 6/60; A61K 6/78

(57) ABSTRACT

A zirconia modification solution and use thereof are provided. The zirconia modification solution includes an opaque component and a coloring component; where a permeability of a mixed system of the opaque component and the coloring component to a dental prosthesis is less than or equal to 20% and a reduction in light transmittance of the dental prosthesis is greater than or equal to 20%; and the opaque component accounts for 30 wt % to 80 wt % of a total mass of the opaque component and the coloring component.

### CROSS REFERENCE TO RELATED APPLICATION

The present application is a continuation-in-part application of International Patent Application No. PCT/CN2024/127719, filed on Oct. 28, 2024, which claims priority to the Chinese Patent Application No. CN202311420248.9, filed with the China National Intellectual Property Administration (CNIPA) on Oct. 30, 2023, and entitled “ZIRCONIA MODIFICATION SOLUTION AND USE THEREOF”. The disclosure of the two applications is incorporated by references herein in their entireties as part of the present application.

### TECHNICAL FIELD

The present disclosure relates to the technical field of dental materials, and in particular relates to a zirconia modification solution and use thereof.

### BACKGROUND

With the improvement in living standards, the demand for prosthodontics has evolved beyond restoring masticatory function to pay more attention to post-restoration aesthetic appearance. The advancement of zirconia ceramic manufacturing technology has facilitated the widespread application of aesthetic ceramics in prosthodontics. The aesthetic requirements for dentures necessitate that restorative materials closely match and harmonizes with natural teeth in both color and translucency. However, the inherent high translucency of aesthetic ceramics presents limitations in scenarios requiring opacity, such as thin-section applications, deep-colored abutment teeth, and restoration of the upper part of an implant. Such an insufficient masking capability allows the color of the underlying abutment teeth or metal abutments to show through, ultimately compromising the overall aesthetic appearance of the dental prosthesis.

Currently available zirconia modification solutions are generally categorized into coating-type and infiltration-type products. Coating-type modification solution products enhance opacity through surface film deposition on an inner surface of a dental crown after final sintering. Yet, these processes involve complex coating techniques, operationally cumbersome workflows, and high costs. Infiltration-type modification solution products are applied prior to the final sintering of prostheses. However, conventional infiltration-type modification solution products exhibit a large infiltration depth, resulting in an unnatural chalky white appearance post-final sintering that becomes visible through the dental crown, significantly compromising restoration effect. Such products fail to achieve aesthetic effects in ultra-thin prostheses particularly like clinical veneers.

CN108703890A has disclosed an opaque zirconia prosthesis formed by applying one or multiple layers selected from a stained opaque layer, a coated opaque layer, and a colored adhesive layer to a fitting surface of the zirconia prosthesis. However, this opaque zirconia prosthesis requires a multi-layer composite opaque masking method to achieve opaque masking effect, with complex processing steps and high dependency on operation technique level, while excessive opaque layers may necessitate excessive tooth preparation during clinical procedures, thereby limiting its practical application and widespread adoption in clinical settings.

CN110314001A has disclosed an opacity-imparting liquid, which is applied to a portion of a highly translucent zirconia dental crown to adjust transparency without coloring. This liquid material, used for an affixing device for cutting from zirconium oxide for dental cutting, includes: (a) 10 wt % to 39 wt % of a water-soluble aluminum compound and/or a water-soluble lanthanum compound; (b) 60 wt % to 89 wt % of water; and (c) 1 wt % to 20 wt % of an organic solvent. However, the high translucency of the entire zirconia crown and its outer layer allows the opaque layer formed on the treated inner surface to visibly show through the outer crown structure, which compromises the overall translucency and color harmony of prosthesis, thereby diminishing both the restorative material quality and clinical restorative effects.

CN109608233A has disclosed a technique for enhancing the translucency of dental zirconia ceramics, specifically involving an opaque liquid. The opaque liquid includes potassium nitrate, yttrium chloride, praseodymium nitrate, alcohols, citric acid, gluconic acid, and water. The opaque liquid is applied to the zirconia surface before final sintering process, and the final sintering is then conducted. However, the opaque liquid improves light transmission, and thus fails to adequately mask darker prepared teeth, discolored teeth, or metal abutments to realize an optimal opaque effect, leading to that the underlying substrate color shows through the dental crown, severely compromising an aesthetic appearance of the prosthesis.

Therefore, in view of the shortcomings of the existing technology, it is urgently necessary to provide a zirconia modification solution that could achieve the opaque effect without impairing the overall aesthetics of a dental prosthesis.

### SUMMARY

An object of the present disclosure is to provide a zirconia modification solution and use thereof. The zirconia modification solution achieves an opaque effect by controlling contents of the opaque component and the coloring component, which enables an opaque layer to effectively conceal dark abutment teeth, discolored teeth, or metal abutments, simultaneously without impairing an aesthetic appearance of the dental prosthesis.

In some embodiments of the present disclosure, a product form of the zirconia modification solution is selected from the group consisting of a separated packaging form and a homogenous form. Products in different forms could all be referred to as zirconia modification solutions. When specifically referring to in the separated packaging form or the homogenous form, they are specifically the separated packaging product form or the homogenous product form.

In order to achieve the object of the present disclosure, the present disclosure adopts the following technical solutions:

In a first aspect, the present disclosure provides a zirconia modification solution, including/consisting of an opaque component and a coloring component; where
a permeability the zirconia modification solution to a dental prosthesis is less than or equal to 20% and a reduction in light transmittance of the dental prosthesis is greater than or equal to 20%. The zirconia modification solution achieves an opaque effect without impairing aesthetics of the dental prosthesis. In the present disclosure, the reduction in light transmittance of the dental prosthesis refers to the light transmittance of the zirconia-based dental prosthesis obtained after opaque masking the dental prosthesis with the zirconia modification solution, relative to the light transmittance of the dental prosthesis before opaque masking.

In some embodiments of the present disclosure, a product form of the zirconia modification solution is selected from the group consisting of a separated packaging form and a homogeneous form; the separated packaging form is obtained by separately packaging the opaque component and the coloring component; and the homogeneous form corresponds to a mixed system of the opaque component and the coloring component

A method for the opaque masking is described in use of the zirconia modification solution described below.

The mixed system of the opaque component and the coloring component includes: based on a total mass of the mixed system being 100 wt %, 30 wt % to 80 wt % of the opaque component and the coloring component as a balance. In the present disclosure, the zirconia modification solution achieves an opaque effect by controlling a proportion of the opaque component to the coloring component, which enables an opaque layer to effectively conceal dark abutment teeth, discolored teeth, or metal abutments, thereby avoiding the showing thereof on a surface of a zirconia-based dental prosthesis, and compromised restorative outcomes. Also, the zirconia modification solution not only achieves an opaque effect, but also does not impair an aesthetic appearance of the dental prosthesis. By concurrently adjusting the opaque layer's color, the formulation avoids too high zirconia translucency that might allow the showing of opaque layer's color to compromise aesthetic outcomes.

The opaque component accounts for 30 wt % to 80 wt %, with exemplary values but not limited to 30 wt %, 35 wt %, 40 wt %, 45 wt %, 50 wt %, 55 wt %, 60 wt %, 65 wt %, 70 wt %, 75 wt %, or 80 wt % of the mixed system composed of the opaque component and the coloring component. Other unlisted values within this range remain equally applicable.

Insufficient content of the opaque component in the mixed system may fail to mask dark abutment teeth, discolored teeth, or metal abutments, allowing their showings on the surface of the zirconia prosthesis and thereby compromising restorative effects. Conversely, excessive content of the opaque component risks the showing of the color of the opaque layer, adversely affecting the aesthetic appearance of the dental prosthesis.

A permeability of the zirconia modification solution to a dental prosthesis is <20%, with exemplary values but not limited to 20%, 18%, 15%, 12%, or 10%; other unlisted values within this range are equally applicable.

The dental prosthesis demonstrates a reduction of not less than 20% in light transmittance, with exemplary values but not limited to 20%, 22%, 25%, 28%, or 30%; any unlisted values within the defined range are equally applicable.

In some embodiments, the opaque component includes a silicon-containing amphiphilic compound.

In some embodiments, the opaque component includes any one or a combination of at least two selected from the group consisting of KH550 (3-aminopropyltriethoxysilane), KH560 (3-glycidoxypropyltrimethoxysilane), KH570 (γ-methacryloyloxypropyl trimethoxysilane), KH792 (N-(2-aminoethyl)-3-aminopropyltrimethoxysilane), and DL602 (N-(2-aminoethyl)-3-aminopropyl methyl dimethoxysilane). Typical but non-limiting combinations include: a combination of KH550 and KH560; a combination of KH560 and KH570; a combination of KH570 and KH792; a combination of KH570 and DL602; a combination of KH792 and DL602; a combination of KH550, KH560 and KH570; a combination of KH560, KH570 and DL602; a combination of KH570, KH792, and DL602; or a combination of KH550, KH560, KH570, KH792, and DL602.

In the present disclosure, the incorporated opaque component synergistically interacts with the coloring component to form a multiphase layer through high-temperature sintering. This composite structure (composite-phase layer) exhibits minimal transparency, achieving the desired opacity effect while ensuring that the zirconia-based dental prostheses meet aesthetic requirements during use. The opaque layer (composite-phase layer) demonstrates a lower penetration depth, and could not penetrate to the outer surface of a zirconia prosthesis, which otherwise adversely affects the aesthetic appearance of the zirconia prosthesis. Furthermore, the adjustable coloration of this opaque layer (composite-phase layer) enables precise simulation of natural dentin hue, thereby achieving enhanced biomimetic aesthetic outcomes.

In some embodiments, the coloring component includes a soluble metal salt, a stabilizer, and a solvent.

In some embodiments, the coloring component includes 0.5 wt % to 58.18 wt % of the soluble metal salt, 0.13 wt % to 16 wt % of the stabilizer, and the solvent as a balance, based on a mass of the coloring component being 100 wt %.

The soluble metal salt has a mass percentage content of 0.5 wt % to 58.18 wt %, with exemplary values but not limited to 0.5 wt %, 1 wt %, 7 wt %, 14 wt %, 20 wt %, 28 wt %, 30 wt %, 35 wt %, 40 wt %, 45 wt %, 50 wt %, or 58.18 wt %; other unlisted values within this range are equally applicable.

The stabilizer has a mass percentage content of 0.13 wt % to 16 wt %, with exemplary values but not limited to 0.13 wt %, 0.5 wt %, 1.6 wt %, 3.2 wt %, 8.18 wt %, 10 wt %, 12 wt %, 14 wt %, 15 wt %, or 16 wt %; other unlisted values within this range are equally applicable.

In some embodiments, a cation in the soluble metal salt includes any one or a combination of at least two selected from the group consisting of Fe³⁺, Ce³⁺, Er³⁺, Nd³⁺, Cr³⁺, Mn²⁺, Al³⁺, CO²⁺, and Gd³⁺. Typical but non-limiting combinations include: a combination of Fe³⁺ and Ce³⁺; a combination of Fe³⁺ and Er³⁺; a combination of Er³⁺, Co²⁺ and Fe³⁺; a combination of Fe³⁺ and Mn²⁺; a combination of Fe³⁺ and Al³⁺; a combination of Fe³⁺, Al³⁺ and Mn²⁺; a combination of Fe³⁺, Ce³⁺ and Cr³⁺; a combination of Co²⁺, Cr³⁺, Er³⁺ and Nd³⁺; a combination of Er³⁺, Nd³⁺ and Cr³⁺; a combination of Nd³⁺, Cr³⁺, Mn²⁺, Co²⁺ and Gd³⁺; or a combination of Fe³⁺, Ce³⁺, Er³⁺, Nd³⁺, Cr³⁺, Mn²⁺, CO²⁺ and Gd³⁺.

In some embodiments, an anion in the soluble metal salt includes any one or a combination of at least two selected from the group consisting of NO₃⁻, CH₃COO⁻, Cl⁻, C₅H₇O₅COO⁻, and SO₄²⁻. Typical but non-limiting combinations include: a combination of NO₃⁻ and CH₃COO⁻; a combination of Cl⁻, C₅H₇O₅COO⁻, and SO₄²⁻; a combination of NO₃⁻ and SO₄²⁻; a combination of Cl⁻ and C₅H₇O₅COO⁻; a combination of NO₃⁻, CH₃COO⁻, and SO₄²⁻; a combination of NO₃⁻ and Cl⁻; a combination of CH₃COO⁻ and Cl⁻; a combination of NO₃⁻, CH₃COO⁻, and Cl⁻; a combination of NO₃⁻, Cl⁻, C₅H₇O₅COO⁻, and SO₄²⁻; or a combination of NO₃⁻, CH₃COO⁻, Cl⁻, C₅H₇O₅COO⁻, and SO₄²⁻.

In some embodiments, the stabilizer includes any one or a combination of at least two selected from the group consisting of citric acid, tartaric acid, lactic acid, oxalic acid, malic acid, ascorbic acid, and polydextrose. Typical but non-limiting combinations include: a combination of citric acid and tartaric acid; a combination of citric acid and polydextrose; a combination of oxalic acid and polydextrose; a combination of tartaric acid and polydextrose; a combination of lactic acid, malic acid and ascorbic acid; a combination of lactic acid and malic acid; a combination of lactic acid, oxalic acid, malic acid, and ascorbic acid; or a combination of citric acid, tartaric acid, lactic acid, oxalic acid, malic acid, ascorbic acid, and polydextrose.

In some embodiments, the solvent includes any one or a combination of at least two selected from the group consisting of deionized water, pentaerythritol, ethylene glycol, 1,2-propanediol, 1,3-butanediol, 1,4-butanediol, polyethylene glycol, 1,6-hexanediol, neopentyl glycol, diethylene glycol, dipropylene glycol (I), trimethylolpropane, and glycerol. Typical but non-limiting combinations include: a combination of deionized water and pentaerythritol; a combination of ethylene glycol, 1,2-propanediol, 1,3-butanediol, 1,4-butanediol, polyethylene glycol, and 1,6-hexanediol; a combination of 1,6-hexanediol, neopentyl glycol, diethylene glycol, dipropylene glycol, trimethylolpropane, and glycerol; or a combination of deionized water, pentaerythritol, ethylene glycol, 1,2-propanediol, 1,3-butanediol, 1,4-butanediol, polyethylene glycol, 1,6-hexanediol, neopentyl glycol, diethylene glycol, dipropylene glycol (I), trimethylolpropane, and glycerol.

The present disclosure further provides a method for preparing the zirconia modification solution in the homogenous form, including: mixing 30 wt % to 80 wt % of the opaque component and the coloring component as the balance, based on a total mass of the zirconia modification solution being 100%.

In a second aspect, the present disclosure provides use of the zirconia modification solution according to the first aspect, where the zirconia modification solution is used for opaque masking of a dental prosthesis; and a method for the opaque masking of the dental prosthesis using the zirconia modification solution includes the following steps:
applying and/or infiltrating the zirconia modification solution onto an inner surface of the dental prosthesis, and then sintering.

The use of the zirconia modification solution effectively reduces internal transparency within dental prostheses, effectively masking dark abutment teeth, discolored teeth, and metal abutment. Featuring a harmonious base color coordinated with the dental prosthesis, it avoids poor aesthetic appearance caused by visible underlying structures through the prosthesis surface. Additionally, by adjusting the coloration of the opaque layer formed from the zirconia modification solution, it addresses potential aesthetic compromises where too high translucency of high-translucency zirconia may allow undesired showing of the color of the opaque layer.

In some embodiments, the sintering is conducted at a temperature of 1,450° C. to 1,570° C., with exemplary values but not limited to 1,450° C., 1,480° C., 1,500° C., 1,530° C., or 1,570° C.; other values not listed within the numerical range are equally applicable.

In some embodiments, the sintering is conducted for 10 min to 150 min, with exemplary values but not limited to 10 min, 50 min, 100 min, 120 min, or 150 min; other values not listed within the numerical range are equally applicable.

Compared with the prior art, embodiments of the present disclosure have the following beneficial effects:

In the present disclosure, the zirconia modification solution includes an opaque component and a coloring component. The zirconia modification solution achieves an opaque effect by controlling contents of the opaque component and the coloring component in the mixed system formed therefrom, which enables the formed opaque layer to effectively conceal dark abutment teeth, discolored teeth, or metal abutments, thereby avoiding the showing thereof on a surface of a zirconia-based dental prosthesis, and compromised restorative outcomes. Also, the zirconia modification solution not only achieves an opaque effect, but also does not impair an aesthetic appearance of the dental prosthesis. By concurrently adjusting the opaque layer's color, the formulation avoids too high zirconia translucency that might allow the showing of opaque layer's color to compromise aesthetic outcomes.

### BRIEF DESCRIPTION OF THE DRAWINGS

FIG. 1 shows an optical image of the zirconia-based dental prosthesis provided in Test Example 3 of the present disclosure;

FIG. 2 shows an optical image of the dental prosthesis provided in Test Example 3 of the present disclosure;

FIG. 3 shows an optical image of the zirconia-based dental prosthesis filled with dark gray plasticine provided in Test Example 3 of the present disclosure; and

FIG. 4 shows an optical image of the dental prosthesis filled with dark gray plasticine provided in Test Example 3 of the present disclosure.

### DETAILED DESCRIPTION OF THE EMBODIMENTS

The technical solutions of the present disclosure will be further described below through specific examples. Those skilled in the art should understand that these examples only help understand the present disclosure and should not be regarded as specific limitations to the present disclosure.

### Example 1

This example provided a zirconia modification solution composed of an opaque component and a coloring component. Based on a total mass of the resulting mixed system being 100 wt %, the zirconia modification solution was composed of 50 wt % of the opaque component (KH550), and the coloring component as a balance. The coloring component was composed of 14 wt % of Fe(NO₃)₃, 3.2 wt % of citric acid, and deionized water as a balance, based on a mass of the coloring component being 100 wt %.

### Example 2

This example provided a zirconia modification solution composed of an opaque component and a coloring component. Based on a total mass of the resulting mixed system being 100 wt %, the zirconia modification solution was composed of 60 wt % of the opaque component (KH560), and the coloring component as a balance. The coloring component was composed of 0.5 wt % of Fe(NO₃)₃, 0.13 wt % of oxalic acid, and ethylene glycol as a balance, based on a mass of the coloring component being 100 wt %.

### Example 3

This example provided a zirconia modification solution composed of an opaque component and a coloring component. Based on a total mass of the resulting mixed system being 100 wt %, the zirconia modification solution was composed of 45 wt % of the opaque component (KH570), and the coloring component as a balance. The coloring component was composed of 58.18 wt % of FeCl₃, 14.18 wt % of lactic acid, and glycerol as a balance, based on a mass of the coloring component being 100 wt %.

### Example 4

This example provided a zirconia modification solution according to Example 1, except that: a mass percentage of the opaque component was adjusted to 30 wt % (with the balance being the coloring component).

### Example 5

This example provided a zirconia modification solution according to Example 1, except that: a mass percentage of the opaque component was adjusted to 80 wt % (with the balance being coloring component).

### Example 6

This example provided a zirconia modification solution according to Example 1, except that: in the coloring component, a mass percentage of Fe(NO₃)₃ was adjusted to 28 wt % and a mass percentage of citric acid was adjusted to 0.08 wt % (with the balance being deionized water).

### Example 7

This example provided a zirconia modification solution according to Example 1, except that: in the coloring component, a mass percentage of Fe(NO₃)₃ was adjusted to 70 wt % and a mass percentage of citric acid was adjusted to 20 wt % (with the balance being deionized water).

### Example 8

This example provided a zirconia modification solution according to Example 1, except that: no citric acid was added to the coloring component, and a mass percentage of the Fe(NO₃)₃ was adaptively adjusted to 50 wt %.

### Example 9

This example provided a zirconia modification solution composed of an opaque component and a coloring component. Based on a total mass of the resulting mixed system being 100 wt %, the zirconia modification solution was composed of 40 wt % of the opaque component (KH570), and the coloring component as a balance. The coloring component was composed of: 16 wt % of FeCl₃, 4.5 wt % of lactic acid, 67 wt % of ethylene glycol, 6 wt % of 1,2-propanediol, 3 wt % of 1,3-butanediol, 3 wt % of 1,4-butanediol, 0.3 wt % of polyethylene glycol, and 0.2 wt % of 1,6-hexanediol, based on a mass of the coloring component being 100 wt %.

### Example 10

This example provided a zirconia modification solution composed of an opaque component and a coloring component. Based on a total mass of the resulting mixed system being 100 wt %, the zirconia modification solution was composed of 30 wt % of the opaque component, and the coloring component as a balance. The opaque component was composed of 50 wt % of KH550 and 50 wt % of KH560, based on a mass of the opaque component being 100 wt %. The coloring component was composed of 14 wt % of Fe(NO₃)₃, 2 wt % of Ce(CH₃COO)₃, 2.2 wt % of citric acid, 1 wt % of tartaric acid, 40.8 wt % of deionized water, and 40 wt % of pentaerythritol, based on a mass of the coloring component being 100 wt %.

### Example 11

This example provided a zirconia modification solution composed of an opaque component and a coloring component. Based on a total mass of the resulting mixed system being 100 wt %, the zirconia modification solution was composed of 80 wt % of the opaque component, and the coloring component as a balance. The opaque component was composed of 40 wt % of DL602, 30 wt % of KH570, and 30 wt % of KH792, based on a mass of the opaque component being 100 wt %. The coloring component was composed of 5 wt % of Cr₂(SO₄)₃, 8 wt % of ErCl₃, 1 wt % of Nd(C₅H₇O₅CO₀)₃, 2.2 wt % of lactic acid, 0.5 wt % of oxalic acid, 0.5 wt % of malic acid, 40 wt % of 1,2-propanediol, 22.8 wt % of 1,3-butanediol, and 20 wt % of 1,4-butanediol, based on a mass of the coloring component being 100 wt %.

### Example 12

This example provided a zirconia modification solution composed of an opaque component and a coloring component. Based on a total mass of the resulting mixed system being 100 wt %, the zirconia modification solution was composed of 50 wt % of the opaque component, and the coloring component as a balance. The opaque component was composed of 50 wt % of KH550, 25 wt % of KH570, 15 wt % of KH792, and 10 wt % of DL602, based on a mass of the opaque component being 100 wt %. The coloring component was composed of 5 wt % of Cr₂(SO₄)₃, 5 wt % of Mn(CH₃COO)₂, 8 wt % of Co(NO₃)₂, 8 wt % of NdCl₃, 2 wt % of Gd(C₅H₇O₅COO)₃, 0.04 wt % of malic acid, 0.04 wt % of ascorbic acid, 30 wt % of 1,6-hexanediol, 20 wt % of neopentyl glycol, 10 wt % of diethylene glycol, 10 wt % of dipropylene glycol, 1 wt % of trimethylolpropane, and 0.92 wt % of glycerol, based on a mass of the coloring component being 100 wt %.

### Example 13

This example provided a zirconia modification solution composed of an opaque component and a coloring component. Based on a total mass of the resulting mixed system being 100 wt %, the zirconia modification solution was composed of 50 wt % of the opaque component, and the coloring component as a balance. The opaque component was composed of 70 wt % of KH550, 10 wt % of KH560, 10 wt % of KH570, 5 wt % of KH792, and 5 wt % of DL602, based on a mass of the opaque component being 100 wt %. The coloring component was composed of 35 wt % of Fe(NO₃)₃, 5 wt % of Cr(NO₃)₃, 5 wt % of Mn(NO₃)₂, 8 wt % of Er(NO₃)₃, 8 wt % of Co(NO₃)₂, 6 wt % of Nd(NO₃)₃, 1 wt % of Ce(NO₃)₃, 2 wt % of Gd(NO₃)₃, 10 wt % of lactic acid, 5 wt % of malic acid, 3 wt % of oxalic acid, 2 wt % of ascorbic acid, 8 wt % of deionized water, 0.5 wt % of pentaerythritol, 0.5 wt % of ethylene glycol, 0.1 wt % of 1,2-propanediol, 0.1 wt % of 1,3-butanediol, 0.1 wt % of 1,4-butanediol, 0.1 wt % of polyethylene glycol, 0.1 wt % of 1,6-hexanediol, 0.1 wt % of neopentyl glycol, 0.1 wt % of diethylene glycol, 0.1 wt % of dipropylene glycol, 0.1 wt % of trimethylolpropane, and 0.1 wt % of glycerol, based on a mass of the coloring component being 100 wt %.

### Example 14

This example provided a zirconia modification solution composed of an opaque component and a coloring component. Based on a total mass of the resulting mixed system being 100 wt %, the zirconia modification solution was composed of 50 wt % of the opaque component, and the coloring component as a balance. The opaque component was composed of 40 wt % of KH560, 60 wt % of KH570, based on a mass of the opaque component being 100 wt %. The coloring component was composed of 14 wt % of Fe₂(SO₄)₃, 3.5 wt % of citric acid, 0.5 wt % of polydextrose, 41 wt % of deionized water, and 41 wt % of 1,3-butanediol, based on a mass of the coloring component being 100 wt %.

### Example 15

This example provided a zirconia modification solution composed of an opaque component and a coloring component. Based on a total mass of the resulting mixed system being 100 wt %, the zirconia modification solution was composed of 60 wt % of the opaque component, and the coloring component as a balance. The opaque component was composed of 70 wt % of KH550, and 30 wt % of KH792, based on a mass of the opaque component being 100 wt %. The coloring component was composed of 1.6 wt % of FeCl₃, 0.3% of Al(NO₃)₃, 0.3 wt % of oxalic acid, 0.2 wt % of polydextrose, 49 wt % of ethylene glycol, and 49 wt % of 1,2-propanediol, based on a mass of the coloring component being 100 wt %.

### Example 16

This example provided a zirconia modification solution composed of an opaque component and a coloring component. Based on a total mass of the resulting mixed system being 100 wt %, the zirconia modification solution was composed of 45 wt % of the opaque component, and the coloring component as a balance. The opaque component was composed of 80 wt % of KH570, 20 wt % of KH792, based on a mass of the opaque component being 100 wt %. The coloring component was composed of 40 wt % of FeCl₃, 18 wt % of ErCl₃, 11.5 wt % of lactic acid, 2.5 wt % of malic acid, 2 wt % of ascorbic acid, 22 wt % of deionized water, 3 wt % of ethanol, and 1 wt % of glycerol, based on a mass of the coloring component being 100 wt %.

### Example 17

This example provided a zirconia modification solution composed of an opaque component and a coloring component. Based on a total mass of the resulting mixed system being 100 wt %, the zirconia modification solution was composed of 40 wt % of the opaque component with the coloring component as a balance. The opaque component was composed of 60 wt % of KH570, 40 wt % of DL602, based on a mass of the opaque component being 100 wt %. The coloring component was composed of 5 wt % of Er(NO₃)₃, 5 wt % of Co(NO₃)₂, 4 wt % of Fe₂(SO₄)₃, 4 wt % of tartaric acid, 62 wt % of deionized water, 10 wt % of diethylene glycol, and 10 wt % of dipropylene glycol, based on a mass of the coloring component being 100 wt %.

### Example 18

This example provided a zirconia modification solution composed of an opaque component and a coloring component. Based on a total mass of the resulting mixed system being 100 wt %, the zirconia modification solution was composed of 70 wt % of the opaque component), and the coloring component as a balance. The opaque component was composed of 80 wt % of KH792, 20 wt % of DL602, based on a mass of the opaque component being 100 wt %. The coloring component was composed of 3 wt % of ErCl₃, 4 wt % of CoCl₂, 7 wt % of Fe(C₅H₇O₅COO)₃, 4 wt % of citric acid, 72 wt % of deionized water, and 10 wt % of polyethylene glycol, based on a mass of the coloring component being 100 wt %.

### Example 19

This example provided a zirconia modification solution composed of an opaque component and a coloring component. Based on a total mass of the resulting mixed system being 100 wt %, the zirconia modification solution was composed of 40 wt % of the opaque component, and the coloring component as a balance. The opaque component was composed of 80 wt % of KH550, 10 wt % of KH560, and 10 wt % of KH570, based on a mass of the opaque component being 100 wt %. The coloring component was composed of 28 wt % of FeCl₃, 10% of Mn(CH₃COO)₂, 11 wt % of malic acid, 42 wt % of ethylene glycol, 3 wt % of 1,2-propanediol, 3 wt % of 1,3-butanediol, and 3 wt % of 1,4-butanediol, based on a mass of the coloring component being 100 wt %.

### Example 20

This example provided a zirconia modification solution composed of an opaque component and a coloring component. Based on a total mass of the resulting mixed system being 100 wt %, the zirconia modification solution was composed of 30 wt % of the opaque component, and the coloring component as a balance. The opaque component was composed of 45 wt % of KH560, 45 wt % of KH570, and 10 wt % of DL602, based on a mass of the opaque component being 100 wt %. The coloring component was composed of 26 wt % of Fe(NO₃)₃, 22 wt % of Ce(CH₃COO)₃, 2 wt % of Cr₂(SO₄)₃, 12 wt % of tartaric acid, 2 wt % of polydextrose, 30 wt % of deionized water, and 6 wt % of polyethylene glycol, based on a mass of the coloring component being 100 wt %.

### Example 21

This example provided a zirconia modification solution composed of an opaque component and a coloring component. Based on a total mass of the resulting mixed system being 100 wt %, the zirconia modification solution was composed of 80 wt % of the opaque component, and the coloring component as a balance. The opaque component was composed of 30 wt % of KH550, 30 wt % of KH560, 30 wt % of KH570, and 10 wt % of KH792, based on a mass of the opaque component being 100 wt %. The coloring component was composed of 15 wt % of Co(NO₃)₂, 8 wt % of Cr₂(SO₄)₃, 5 wt % of ErCl₃, 0.5 wt % of Nd(C₅H₇O₅COO)₃, 12.5 wt % of lactic acid, 1 wt % of malic acid, 38 wt % of 1,3-butanediol, 10 wt % of 1,4-butanediol, and 10 wt % of glycerol, based on a mass of the coloring component being 100 wt %.

### Example 22

This example provided a zirconia modification solution composed of an opaque component and a coloring component. Based on a total mass of the resulting mixed system being 100 wt %, the zirconia modification solution was composed of 50 wt % of the opaque component (KH570), and the coloring component as a balance. The coloring component was composed of 1.2 wt % of FeCl₃, 0.2% of Al(NO₃)₃, 0.2% of Mn(CH₃COO)₂, 1 wt % of citric acid, 48.7 wt % of deionized water, and 48.7 wt % of 1,3-butanediol, based on a mass of the coloring component being 100 wt %.

### Comparative Example 1

This comparative example provided a zirconia modification solution according to Example 1, except that a mass percentage of the opaque component was adjusted to 20 wt % (with the balance being the coloring component).

### Comparative Example 2

This comparative example provided a zirconia modification solution according to Example 1, except that a mass percentage of the opaque component was adjusted to 90 wt % (with the balance being the coloring component).

### Comparative Example 3

This comparative example provided a dental prosthesis, where the dental prosthesis was obtained by only sintering at 1,480° C. for 100 min.

### Test Example 1

Permeability was tested by immersing 15 mm×10 mm×8 mm dental prostheses in the zirconia modification solutions provided in Examples 1 to 13 and Comparative Examples 1 to 2 for 1 s, followed by taking out and surface wiping to eliminate excess liquid. The dental prostheses samples were air-dried at room temperature for 10 min and subsequently oven-dried at 120° C. for 30 min. Each dental prosthesis was then bisected along its long axis using a grinding bur, and the cross-sectional surfaces obtained thereby were sequentially polished using 800-mesh and 2000-mesh sandpaper until plain. The bisected samples underwent sintering at 1,480° C. for 100 min with their polished surfaces facing downward to produce zirconia-based dental prostheses. Permeation characteristics were analyzed using high-definition microscopy, with calculated permeability shown in Table 1. The permeability was calculated according to the following equation:

$$the permeability = \frac{penetration depth}{2 \times the total length ( or width )} \times 100 \% .$$

### Test Example 2

Light transmittance was tested by applying the zirconia modification solutions provided in Examples 1 to 13 and Comparative Examples 1 to 2 to one surface of 15 mm×10 mm×8 mm dental prostheses. The coated dental prosthesis samples were air-dried at room temperature for 10 min, oven-dried at 120° C. for 5 min, then flipped and dried for an additional 5 min. After another flip, they were dried at 180° C. for 10 min, followed by taking out and then sintering at 1,480° C. for 100 min to produce zirconia-based dental prostheses. Light transmittance was measured using a haze meter. Comparative Example 3 served as a control without applying the zirconia modification solution, with its light transmittance similarly measured. The reductions in light transmittance of Examples 1 to 12 and Comparative Examples 1 to 2 relative to Comparative Example 3 were calculated, with results shown in Table 1.

### Test Example 3

Opacity, aesthetic quality, and solution stability evaluations were conducted by uniformly applying the zirconia modification solutions provided in Examples 1 to 13 and Comparative Examples 1 to 2 to an inner surface of 1.2 mm-thick anterior tooth prostheses. Samples were air-dried at room temperature for 10 min, oven-dried at 120° C. for 10 min, and sintered at 1,480° C. for 100 min to produce zirconia-based dental prostheses. Opaque masking performance was assessed by filling dental prostheses with dark gray plasticine (simulating discolored abutment teeth or metal abutments) and comparing with Comparative Example 3. An outer surface of a prosthesis appearing no obvious color of dark abutment teeth in the prosthesis was rated as having good masking ability, otherwise classified as poor. Aesthetic quality was determined by comparing surface color transmission with Comparative Example 3, An outer surface of a dental prosthesis appearing no obvious color of the opaque layer on the inner surface of the dental prosthesis was deemed aesthetically superior, otherwise deemed poor. Solution stability was evaluated by monitoring sealed zirconia modification solutions over 10 min; formulations exhibiting flocculation or precipitation were considered unstable, while those maintaining homogeneity were classified as stable. Results for all the assessments are summarized in Table 1.

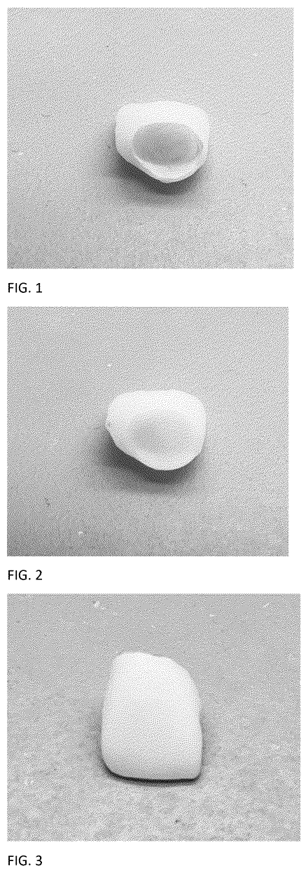

Optical images of the zirconia-based dental prostheses in Example 1 and Comparative Example 3 are shown in FIG. 1 and FIG. 2, respectively. The zirconia-based dental prostheses coated with the zirconia modification solution develop a color-effect opaque layer on their inner surfaces, and the color of the opaque layer does not show on an outer surface of the dental prosthesis, which substantially does not affect the color of the outer surface of the dental prosthesis, thereby demonstrating superior aesthetic performance. In contrast, a dental prosthesis not coated with the zirconia modification solution exhibits no significant differences in optical characteristics between their inner and outer surfaces, preserving favorable aesthetics but lacking any opaque masking capability.

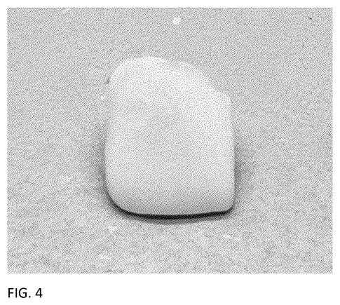

Optical evaluation of the zirconia-based dental prosthesis in Example 1 filled with dark gray plasticine (FIG. 3) demonstrate effective opaque masking performance, with no discernible color transmission from the simulated dark abutment teeth in the dental prosthesis to the external surface thereof. Conversely, optical evaluation of the dental prosthesis in Comparative Example 3 filled with dark gray plasticine is shown in FIG. 4, the color of the simulated dark abutment teeth in the dental prosthesis obviously shows on the external surface of the dental prosthesis, exhibiting inadequate opaque masking capability.

TABLE 1

| | | Reduction | | | |
|---|---|---|---|---|---|
| | Perme- | in light | | | |
| | ability | transmittance | | Aesthetic | Solution |
| Name | (%) | (%) | Opacity | quality | stability |
| Example 1 | 11.00 | 28.53 | Good | Good | Stable |
| Example 2 | 7.17 | 22.77 | Good | Good | Stable |
| Example 3 | 18.28 | 67.16 | Good | Good | Stable |
| Example 4 | 19.24 | 26.77 | Good | Good | Stable |
| Example 5 | 19.95 | 32.74 | Good | Good | Stable |
| Example 6 | 16.84 | 30.04 | Good | Poor | Unstable |
| Example 7 | 20.00 | 71.41 | Good | Poor | Unstable |
| Example 8 | 18.21 | 59.87 | Good | Good | Unstable |
| Example 9 | 15.63 | 27.65 | Good | Good | Stable |
| Example 10 | 19.05 | 25.61 | Good | Good | Stable |
| Example 11 | 19.78 | 31.23 | Good | Good | Stable |
| Example 12 | 15.33 | 32.35 | Good | Poor | Unstable |
| Example 13 | 19.96 | 69.87 | Good | Poor | Unstable |
| Example 14 | 10.57 | 27.96 | Good | Good | Stable |
| Example 15 | 8.72 | 25.16 | Good | Good | Stable |
| Example 16 | 18.04 | 65.75 | Good | Good | Stable |
| Example 17 | 18.61 | 26.01 | Good | Good | Stable |
| Example 18 | 19.34 | 35.62 | Good | Good | Stable |
| Example 19 | 15.21 | 28.05 | Good | Good | Stable |
| Example 20 | 17.25 | 28.26 | Good | Good | Stable |
| Example 21 | 19.33 | 32.97 | Good | Good | Stable |
| Example 22 | 9.86 | 23.64 | Good | Good | Stable |
| Comparative | 22.00 | 18.62 | Good | Poor | Stable |
| Example 1 | | | | | |
| Comparative | 25.00 | 34.59 | Good | Poor | Stable |
| Example 2 | | | | | |
| Comparative | / | / | Poor | Poor | / |
| Example 3 | | | | | |

Analysis of Table 1 demonstrates that the zirconia modification solution proposed effectively achieves dental prosthesis opaque masking while preventing aesthetic compromise caused by chromatic transmission of the opaque layer through a surface of a zirconia-based prosthesis.

The comparisons between Examples 1 to 5, 10 to 11, and Comparative Examples 1 to 2 demonstrate that the contents of the opaque component and the coloring component in the zirconia modification solution need to be defined within appropriate ranges. Insufficient or excessive opaque component content results in poor opaque masking performance or showing through of the color of the opaque layer (permeability exceeding 2000 causes result in the color of the opaque layer showing through), thereby compromising aesthetic outcomes. From comparisons between Example 1 and Examples 6, 7, 12, and 13, the contents of the soluble metal salt and the stabilizer in the coloring component also need to be controlled in rational ranges, otherwise, the excessively white color of the opaque layer shows through the prosthesis, thereby resulting in increased lightness of the prosthesis, or the color of the opaque layer shows through the prosthesis, resulting in unnatural color of the prosthesis, both detrimental to the aesthetics of zirconia-based dental prostheses. The comparison between Example 1 and Example 8 shows that omitting stabilizer in the coloring component induces cationic hydrolysis reactions, generating flocculent precipitates that impair both opaque masking effectiveness and color uniformity.

Comparison between Example 1 and Comparative Example 3 confirms that not applying the zirconia modification solution onto the inner surface of the dental prosthesis could not achieve the opaque masking effect.

In conclusion, the proposed zirconia modification solution achieves an opaque effect by controlling contents of the opaque component and the coloring component, which enables an opaque layer to effectively conceal dark abutment teeth, discolored teeth, or metal abutments, thereby avoiding their showing on the outer surface of the zirconia-based dental prosthesis, and compromised restorative outcomes. Also, the zirconia modification solution not only achieves an opaque effect, but also does not impair an aesthetic appearance of the dental prosthesis. By concurrently adjusting the opaque layer's color, the formulation avoids too high zirconia translucency that might allow the showing of opaque layer's color to compromise aesthetic appearance.

The above are merely specific embodiments of the present disclosure, and the scope of the present disclosure is not limited thereto. Those skilled in the art should understand that any modification or replacement easily conceived by those skilled in the art within the technical scope of the present disclosure should fall within the scope of the present disclosure.

What is claimed is:

1. A zirconia modification solution, comprising an opaque component and a coloring component; wherein
a permeability of the zirconia modification solution to a dental prosthesis is less than or equal to 20%, and a reduction in light transmittance of the dental prosthesis is greater than or equal to 20%; and
the opaque component accounts for 30 wt % to 80 wt % of a total mass of the opaque component and the coloring component.

2. The zirconia modification solution as claimed in claim 1, wherein a product form of the zirconia modification solution comprises one selected from the group consisting of a separated packaging form and a homogeneous form;
the separated packaging form is obtained by separately packaging the opaque component and the coloring component; and
the homogeneous form corresponds to the mixed system of the opaque component and the coloring component.

3. The zirconia modification solution as claimed in claim 1, wherein the zirconia modification solution achieves an opaque effect without impairing aesthetics of the dental prosthesis.

4. The zirconia modification solution as claimed in claim 1, wherein the opaque component comprises a silicon-containing amphiphilic compound.

5. The zirconia modification solution as claimed in claim 1, wherein the opaque component comprises at least one selected from the group consisting of 3-aminopropyltriethoxysilane, 3-glycidoxypropyltrimethoxysilane, γ-methacryloyloxypropyl trimethoxysilane, N-(2-aminoethyl)-3-aminopropyltrimethoxysilane, and N-(2-aminoethyl)-3-aminopropyl methyl dimethoxysilane.

6. The zirconia modification solution as claimed in claim 5, wherein the opaque component comprises one selected from the group consisting of: a combination of the 3-aminopropyltriethoxysilane and the 3-glycidoxypropyltrimethoxysilane; a combination of the 3-glycidoxypropyltrimethoxysilane and the γ-methacryloyloxypropyl trimethoxysilane; a combination of the γ-methacryloyloxypropyl trimethoxysilane and the N-(2-aminoethyl)-3-aminopropyltrimethoxysilane; a combination of the γ-methacryloyloxypropyl trimethoxysilane and the N-(2-aminoethyl)-3-aminopropyl methyl dimethoxysilane; a combination of the N-(2-aminoethyl)-3-aminopropyltrimethoxysilane and the N-(2-aminoethyl)-3-aminopropyl methyl dimethoxysilane; a combination of the 3-aminopropyltriethoxysilane, the 3-glycidoxypropyltrimethoxysilane and the γ-methacryloyloxypropyl trimethoxysilane; a combination of the 3-glycidoxypropyltrimethoxysilane, the γ-methacryloyloxypropyl trimethoxysilane, and the N-(2-aminoethyl)-3-aminopropyl methyl dimethoxysilane; a combination of the γ-methacryloyloxypropyl trimethoxysilane, the N-(2-aminoethyl)-3-aminopropyltrimethoxysilane, and the N-(2-aminoethyl)-3-aminopropyl methyl dimethoxysilane; and a combination of the 3-aminopropyltriethoxysilane, the 3-glycidoxypropyltrimethoxysilane, the γ-methacryloyloxypropyl trimethoxysilane, the N-(2-aminoethyl)-3-aminopropyltrimethoxysilane and the N-(2-aminoethyl)-3-aminopropyl methyl dimethoxysilane.

7. The zirconia modification solution as claimed in claim 1, wherein the coloring component comprises a soluble metal salt, a stabilizer, and a solvent.

8. The zirconia modification solution as claimed in claim 7, wherein the coloring component comprises 0.5 wt % to 58.18 wt % of the soluble metal salt, 0.13 wt % to 16 wt % of the stabilizer, and the solvent as a balance, based on a mass of the coloring component being 100 wt %.

9. The zirconia modification solution as claimed in claim 7, wherein a cation in the soluble metal salt comprises at least one selected from the group consisting of Fe³⁺, Ce³⁺, Er³⁺, Nd³⁺, Cr³⁺, Mn²⁺, Al³⁺, Co²⁺, and Gd³⁺.

10. The zirconia modification solution as claimed in claim 9, wherein the cation in the soluble metal salt comprises one selected from the group consisting of: a combination of the Fe³⁺ and the Ce³⁺; a combination of the Fe³⁺ and the Er³⁺; a combination of the Er³⁺, the Co²⁺, and the Fe³⁺; a combination of the Fe³⁺ and the Mn²⁺; a combination of the Fe³⁺ and the Al³⁺; a combination of the Fe³⁺, the Al³⁺, and the Mn²⁺; a combination of the Fe³⁺, the Ce³⁺, and the Cr³⁺; a combination of the CO²⁺, the Cr³⁺, the Er³⁺, and the Nd³⁺; a combination of the Er³⁺, the Nd³⁺, and the Cr³⁺; a combination of the Nd³⁺, the Cr³⁺, the Mn²⁺, the Co²⁺, and the Gd³⁺; and a combination of the Fe³⁺, the Ce³⁺, the Er³⁺, the Nd³⁺, the Cr³⁺, the Mn²⁺, the Co²⁺, and the Gd³⁺.

11. The zirconia modification solution as claimed in claim 7, wherein an anion in the soluble metal salt comprises at least one selected from the group consisting of NO₃⁻, CH₃COO⁻, Cl⁻, C₅H₇O₅COO⁻, and SO₄²⁻.

12. The zirconia modification solution as claimed in claim 11, wherein the anion in the soluble metal salt comprises one selected from the group consisting of: a combination of the NO₃⁻ and the CH₃COO⁻; a combination of the Cl⁻, the C₅H₇O₅COO⁻, and the SO₄²⁻; a combination of the NO₃⁻ and the SO₄²⁻; a combination of the NO₃⁻ and the Cl⁻; a combination of the CH₃COO⁻ and the Cl⁻; a combination of the NO₃⁻, the CH₃COO⁻, and the Cl⁻; a combination of the Cl⁻ and the C₅H₇O₅COO⁻; a combination of the NO₃⁻, the CH₃COO⁻, and the SO₄²⁻; a combination of the NO₃⁻, the Cl⁻, the C₅H₇O₅COO⁻, and the SO₄²⁻; and a combination of the NO₃⁻, the CH₃COO⁻, the Cl⁻, the C₅H₇O₅COO⁻, and the SO₄²⁻.

13. The zirconia modification solution as claimed in claim 7, wherein the stabilizer comprises at least one selected from the group consisting of citric acid, tartaric acid, lactic acid, oxalic acid, malic acid, ascorbic acid, and polydextrose.

14. The zirconia modification solution as claimed in claim 13, wherein the stabilizer comprises one selected from the group consisting of: a combination of the citric acid and the tartaric acid; a combination of the citric acid and the polydextrose; a combination of the oxalic acid and the polydextrose; a combination of the tartaric acid and the polydextrose; a combination of the lactic acid, the malic acid, and the ascorbic acid; a combination of the lactic acid and the malic acid; a combination of the lactic acid, the oxalic acid, the malic acid, and the ascorbic acid; and a combination of the citric acid, the tartaric acid, the lactic acid, the oxalic acid, the malic acid, the ascorbic acid, and the polydextrose.

15. The zirconia modification solution as claimed in claim 7, wherein the solvent comprises at least one selected from the group consisting of deionized water, pentaerythritol, ethylene glycol, 1,2-propanediol, 1,3-butanediol, 1,4-butanediol, polyethylene glycol, 1,6-hexanediol, neopentyl glycol, diethylene glycol, dipropylene glycol, trimethylolpropane, and glycerol.

16. The zirconia modification solution as claimed in claim 15, wherein the solvent comprises one selected from the group consisting of: a combination of the deionized water and the pentaerythritol; a combination of the ethylene glycol, the 1,2-propanediol, the 1,3-butanediol, the 1,4-butanediol, the polyethylene glycol, and the 1,6-hexanediol; a combination of the 1,6-hexanediol, the neopentyl glycol, the diethylene glycol, the dipropylene glycol, the trimethylolpropane, and the glycerol; and a combination of the deionized water, the pentaerythritol, the ethylene glycol, the 1,2-propanediol, the 1,3-butanediol, the 1,4-butanediol, the polyethylene glycol, the 1,6-hexanediol, the neopentyl glycol, the diethylene glycol, the dipropylene glycol, the trimethylolpropane, and the glycerol.

17. A method for preparing the zirconia modification solution in the homogenous form as claimed in claim 2, comprising:
mixing 30 wt % to 80 wt % of the opaque component and the coloring component as the balance, based on a total mass of the zirconia modification solution being 100%.

18. A method of opaque masking of a dental prosthesis using the zirconia modification solution as claimed in claim 1, comprising:
providing a mixed system of the opaque component and the coloring component, wherein the mixed system comprises, based on the total mass of the mixed system being 100 wt %, 30 wt % to 80 wt % of the opaque component and the coloring component as the balance; and
applying and/or infiltrating the mixed system onto an inner surface of the dental prosthesis, and then sintering.

19. The method as claimed in claim 18, wherein the sintering is conducted at a temperature of 1,450° C. to 1,570° C. for 10 minutes to 150 minutes.

20. A zirconia-based dental prosthesis, comprising a dental prosthesis and an opaque layer arranged on an inner surface of the dental prosthesis; wherein the opaque layer is formed by sintering the zirconia modification solution as claimed in claim 1.
